(19) United States
(12) Patent Application Publication
(10) Pub. No.: US 2024/0200030 A1
(43) Pub. Date: Jun. 20, 2024

(54) TUMOR PROXIMAL CELL COLLECTION

(71) Applicant: DROPLET BIOSCIENCES, INC., Melrose, MA (US)

(72) Inventors: Wendy Winckler Adamovich, Melrose, MA (US); Theresa Tribble, Melrose, MA (US)

(21) Appl. No.: 18/542,322

(22) Filed: Dec. 15, 2023

Related U.S. Application Data
(60) Provisional application No. 63/503,624, filed on May 22, 2023.
(60) Provisional application No. 63/433,388, filed on Dec. 16, 2022.

(51) Int. Cl. C12N 5/0783

(52) U.S. Cl. CPC C12N 5/0636; C12N 2501/2302; C12N 2501/999; C12N 2502/1157

(57) ABSTRACT

The invention provides methods of preparing a cell therapy by collecting fluid from a site proximal to a tumor and expanding T cells obtained from the fluid. Methods include collecting fluid from a site proximal to a tumor, obtaining at least one T cell from the fluid, and culturing the T cells in vitro. The fluid may be collected during or after surgical removal of a tumor from the site of the surgical removal of the tumor. The fluid may be drain fluid that drains from a surgical site. The method may include diverting the fluid from a waste disposal protocol or vessel in a surgical theater and into a collection vessel. Preferably the fluid includes lymphatic fluid.

### TECHNICAL FIELD

The disclosure relates to obtaining T-cells that specifically target tumor cells for use in treating cancer.

### BACKGROUND

Cancer is a leading cause of death globally. Early detection, while beneficial for most cancers, is often difficult. In part, this is because many cancers first develop without presenting any specific clinical symptoms, and diagnosis only occurs when the disease has reached a stage when it is difficult to treat.

Chimeric antigen receptor (CAR) T cell (CAR-T cell) therapy has been a promising approach to personalized immunotherapy for human cancer. CAR-T therapy uses genetic modification to engineer T cells to target tumor-specific antigens, attack specific cancer cells, and bypass tumor cell apoptosis avoidance mechanisms to some extent. CAR-T therapy has been used to treat hematologic diseases but has shown poor results in treating solid tumors. Tumor antigen escape, treatment-related toxicity, and the immunosuppressive tumor microenvironment (TME) are barriers to CAR-T therapy. See Qu, 2022, Tumor buster—where will the CAR-T cell therapy ‘missile’ go? Mol Cancer 21:a201, incorporated by reference.

### SUMMARY

The invention provides methods of preparing a cell therapy by collecting fluid from a site proximal to a tumor and expanding T cells obtained from the fluid. The fluid may be surgical effluent or drain fluid, such as a mixture of lymphatic fluid with blood, interstitial fluid, and/or saline wash that drains from a surgical site. The fluid is obtained from a site that is proximal to the tumor such as the location in a body from which a tumor has been surgically removed. An insight of the disclosure is that, by its proximity to the tumor, the fluid is a natural source of T cells that are already specific to the tumor. Use of effluent from a site proximal to the tumor avoid problems with conventional techniques that require finding vanishingly rare tumor-associated T cells in peripheral blood or trying to liberate intact and viable T cells from within a solid tumor via mechanical and enzymatic processing. Instead, effluent fluids may drain or weep from a surgical site proximal to a tumor location are a source of viable T cells, already trained to specifically recognize tumor cells, that can be cultured in a primary expansion according to methods of the invention, to yield a product rich in (hundreds of thousands of, millions of, or even billions of) expanded, anti-tumor T cells that can be administered back to the patient to treat the cancer by killing any residual tumor cells, such as may be found in surgical margins or metastasized from the original site.

Thus, the invention provides methods and compositions that include an expanded population of autologous T cells for use as an anti-cancer therapy. Compositions and methods of the invention are particularly useful during and after surgical resection of the tumor. The surgery creates a surgical site. Fluid effluent from that site is now recognized and a valuable source of anti-tumor T cells that specifically attack cells descendent from the tumor being removed. T cells can be obtained (e.g., isolated) from that effluent from the site proximal to the tumor and rapidly expanded (e.g., with stimulatory cytokines such as IL-2 and/or feeder cells such as peripheral blood monocytes). The product of that expansion is an in vitro population of T cells that is precision trained to target and kill any tumor cells residual or recurring in the patient after tumor resection. The product can be packaged (e.g., in an IV bag) and delivered back to the patient to kill off any remaining or cryptic tumor cells, such as in the surgical margins or circulating metastatic tumor cell progeny. By those effects, methods and compositions of the invention arrest and prevent cancer recurrence, promoting the patient's chances of a full recovery after cancer therapy.

In fact, for certain patients, methods of the invention may be used as a primary cancer therapy. Fluid may be obtained from a situs proximal to a tumor (e.g., by a surgical incision or using suction into a syringe). The effluent from the situs proximal to the tumor will typically include a rich amount of lymphatic fluid as well as possibly amounts of blood, serum, and/or interstitial fluid. The situs is preferably proximal to (with a few, or ten, or a couple of dozen centimeters of) a tumor. A lymphatic component of that fluid will include, due to the body's natural immune response, T cells that already specifically target tumor cells from that proximal tumor. Even if the effluent fluid from the situs proximal to the tumor only has a small number of anti-tumor T cells, even vanishingly small, even only one, methods of the invention culture and expand that one or more T cells into a rich population of T cells. That product is useful for, and used for, treating the cancer to specifically attack and kill tumor cells of the tumor.

In certain aspects, the invention provides methods of obtaining therapeutic cells. Methods include collecting fluid from a site proximal to a tumor, obtaining at least one T cell from the fluid, and culturing the T cells in vitro. The fluid may be collected during or after surgical removal of a tumor from the site of the surgical removal of the tumor. The fluid may be drain fluid that drains from a surgical site. The method may include diverting the fluid from a waste disposal protocol or vessel in a surgical theater and into a collection vessel. Preferably the fluid includes lymphatic fluid.

In certain embodiments, obtaining the T cell includes an isolation step. For example, the isolation step may include centrifugation, fluorescence-activated cell sorting, capture to magnetic beads, or filtering.

In some embodiments, the culturing step includes an initial expansion with interleukin-2 (IL-2). The culturing step may include a primary expansion of T cells in the presence of IL-2 and irradiated allogeneic peripheral blood mononuclear cells (PBMCs) as feeder cells. The culturing step may additionally or alternatively include a primary expansion or an auxiliary expansion that exposes T cells to one or a combination of a cytokine, costimulatory molecule, immune-checkpoint inhibitor, antigen-presenting cells (APCs), PD-1 blockade, 4-1BB stimulation, and agonistic anti-4-1BB antibody. The culturing step may further include a CD8+ T-cell enrichment using anti-OX40 antibodies. The culturing step may include an expansion phase in the presence of an HLA-matched tumor line.

During or after the culturing, or expansion, methods may include a selection step to select T cells. The selection step may be performed to remove tumor cells from culture. The selection step may also be performed to remove suboptimal T cells, for example, those showing markers of exhaustion such as inhibitory receptors (e.g., CTLA-4, PD-1, BTLA, TIM-3, LAG-3, or TIGIT). Optionally, the selection step uses one or more of laser ablation, flow cytometry, or filtering cells through a membrane. Methods preferably include a quality control process. For example, QC may include a personalized tumor-reactivity assessment that includes the co-culture of T cells with autologous tumor cells.

Methods of the invention may include packaging a product of the culturing step for therapeutic delivery, wherein the product includes 10 to 200 billion T cells. For example, billions of T cells produced by the culturing step may be packaged in an IV bag or sample tube. The product may be held in a cooler, or freezer, or on ice or dry ice, in a hospital or clinical facility. In certain therapeutic treatment embodiments, the methods include administering T cells produced by the culturing step to a patient from whom the fluid was obtained to treat cancer. The administration may be intravenous, intrapleural, intraperitoneal, intrathoracic, intratumoral, or through a surgical drain port. The treatment may be performed to treat head and neck cancer, melanoma, ovarian cancer, breast cancer, colorectal cancer, liver cancer, renal cell carcinoma (RCC), prostate cancer, urothelial bladder carcinoma, gastric carcinoma, gynecologic cancer, or urological cancer.

In another aspect of the invention, methods are provided for post-surgical evaluation of tumor-infiltrating lymphocytes (TILs). Conventionally, levels of circulating TILs are thought to be quite low (e.g., less than about 1%). However, according to the invention, it has been discovered that the TIL fraction in blood is significantly higher post-surgical resection than would be conventionally expected. Without being bound by theory, the invention contemplates that surgical resection of a tumor, and its associated debris, provide antigenic stimulation of TILs in both the surgical drain fluid and, more globally, in peripheral blood. The TIL fraction in peripheral blood post-surgery is useful as a source of therapeutic cells that may be harvested from blood via a venous draw or other procedure (e.g., leukophoresis).

In a preferred aspect a TIL fraction is isolated from peripheral blood between about 3 hours and about 48 hours after a surgical resection. In addition to being a source of therapeutic cells, the TILs also are useful to provide insight into diagnostics, therapeutic selection, and the likelihood of recurrence (primarily based on the type and abundance of TILs in the blood). In addition, TILs are isolated in the drain fluid during or immediately following surgery. Drain fluid TIL fraction is useful as a source of both diagnostic and therapeutic content and its comparison to peripheral blood content is also informative as to both therapy and various aspects of diagnostics.

In a related aspect, the invention provides methods comprising determining a repertoire of TILs in drain fluid and peripheral blood (e.g., in peripheral blood mononuclear cells). In one aspect, the number of clones in drain fluid are compared with the relative number of clones in peripheral blood to determine the percent overlap between drain fluid and blood. It is known that the numbers in drain fluid are generally high due to proximity to the tumor. If the percent overlap between clones in the drain fluid and peripheral blood is high, there is evidence that the surgical intervention had an impact on the repertoire of TILs in peripheral blood, thus leading to tumor-specific therapies and diagnostics as described above.

### BRIEF DESCRIPTION OF THE DRAWINGS

FIG. 1 shows TCR overlap for lymph (drain fluid) versus tissue samples.

### DETAILED DESCRIPTION

The invention uses fluid collected or obtained in proximity to a tumor or site of a tumor. Fluid may be obtained from a situs proximal to a tumor (e.g., by a surgical incision or using suction into a syringe). The effluent from the situs proximal to the tumor will typically include a rich amount of lymphatic fluid as well as possibly amounts of blood, serum, and/or interstitial fluid. The situs is preferably proximal to (with a few, or ten, or a couple of dozen centimeters of) a tumor. A lymphatic component of that fluid will include, due to the body's natural immune response, T cells that already specifically target tumor cells from that proximal tumor. Even if the effluent fluid from the situs proximal to the tumor only has a small number of anti-tumor T cells, even vanishingly small, even only one, methods of the invention culture and expand that one or more T cells into a rich population of T cells.

Lymphatic fluid contains tumor biomarkers that can be interrogated to detect and predict the recurrence, presence, and/or aggressiveness of a tumor. Here, fluid contains one or more anti-tumor T cells that are expanded for therapeutic use. Lymphatic fluid is included in fluid that drains or is irrigated or removed from a surgical site. For example, a tumor resection or a lymphadenectomy (or lymph node dissection) may be performed. Certain types of cancer have a tendency to produce lymph node metastasis, a phenomenon particularly characteristic of melanoma, head and neck cancer, differentiated thyroid cancer, breast cancer, lung cancer, gastric cancer and colorectal cancer. For lymph node dissection, an incision is made in the skin near the affected lymph nodes. The lymph nodes and typically nearby lymphatic tissue and underlying soft tissue and removed. During, and after, such a surgical removal, fluid drains from the surgical site. For a tumor resection, an incision is made, and the tumor is surgically removed. In both examples, fluid drains from the surgical site and that fluid may be referred to as drain fluid. The composition of that drain fluid may vary over time (e.g., may include saline during a surgery when saline is used to irrigate and wash the site), but the drain fluid will reliably include lymph or lymphatic fluid, typically along with blood and interstitial fluid. The lymphatic system regulates immune responses to pathogens and cancer and is characterized by a circulating fluid, lymphatic fluid or lymph, that circulates through the lymphatic system separately from the bloodstream. Lymph is a proximal source of lymphocytes, proteins, and other biomarkers. Methods of the invention involve collecting and stabilizing lymph from surgical drain fluid, which is then useful as a source of T cells. Drain fluid is distinct from blood. Drain fluid typically includes blood (sanguineous or serosanguinous fluids) but also interstitial and lymphatic fluid.

The fluid that drains from the site of such a surgery may include both fluids originating in the patient and any wash used to irrigate the site, such as sterile saline. The surgical drain fluid may typically include different contributing materials including, for example, blood and lymph. However, compared to a venous blood draw, that fluid will include a rich amount of lymphatic fluid and T cells that are already specific to a tumor. Due to the relationship between the surgery and its purpose, fluid that drains from the surgical site has the potential to be rich in material (T cells) that specific to the anatomical target of the surgery (the tumor) and the surrounding tissue. For example, when a tumor resection is performed to remove a tumor, fluid that drains from the site from which the tumor was removed may be rich in material from the proximal tumor or its tumor microenvironment.

Here, the invention uses lymphatic fluid and T cells found within the fluid as a therapeutic. A particular insight of the invention is that lymph contains T-cells and biomarkers of T-cells and, when surgical drain fluid is collected from the site of a surgery such as a tumor resection or lymphadenectomy proximal to a tumor, the T-cells, products thereof, and other tumor biomarkers in the drain fluid are specific to, and have the ability to target, the tumor and cells originating therefrom.

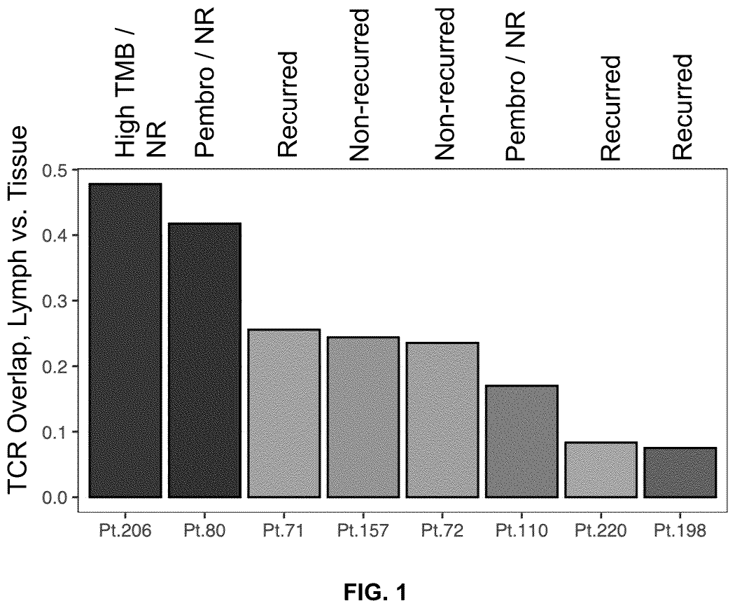

FIG. 1 shows TCR overlap for lymph (drain fluid) versus tissue samples, after controlling for sequencing depth. A number of clones were found in both the blood and the tissue sample from each subject. Controlling for sequencing depth was done by restricting to only the top 1,000 most abundant clones in each sample and identifying shared clones among these subsets. Patients Pt.80, Pt.206, and Pt.71 exhibit the most of the top 1,000 clones shared between samples. This graph demonstrates that that there is a high proportion of T cells in drain fluid.

Once fluid is collected or obtained from a situs proximal to a tumor, methods include obtaining or isolating at least one T cell from the fluid. T cells may be isolated via density gradient centrifugation, e.g., with Ficoll, or specific cell sorting methods, such as magnetic- or fluorescence-activated cell sorting (MACS/FACS). Upon fluid collection, CD8+ T cells could be enriched, or Tregs could be depleted to enhance the antitumor effects of the collected T cells. Some embodiments use the selection of T cells with magnetic beads, such as by selecting CD3+ T cells with Dynabeads (Invitrogen, Thermo Fisher Scientific). Magnetic beads decorated with antibodies against T cell markers (such as CD3) can be used to capture T cells. The beads can be held on a magnet while other components of the fluid (cellular debris, proteins, tumor cells, etc.) are washed away.

Once the initial one or more T cells are obtained, the one or more T cells are expanded. Method may include (optionally) distinct initial and primary expansion phases, or one primary expansion phase. In some embodiments, the cells are subject to an optional initial expansion with high-dose interleukin-2 (IL-2), e.g., for a day or a few days or about a week or two. Methods optionally include performing an initial expansion protocol in vitro using, for example, a rapid expansion protocol. In the initial primary expansion, T cells undergo expansion optionally in the presence of high-dose interleukin-2 (IL-2). For the initial expansion, high dose IL-2, anti-CD3, and irradiated allogeneic peripheral blood mononuclear cells (PBMCs) may be added to the culture as feeder cells. After an initial expansion, a selection step is optionally performed.

Selection steps may be performed at any stage of the overall protocol including at the very beginning, between an initial and primary expansion, during an initial expansion, during a primary expansion, after a primary expansion, during a QC step, after a QC step or at any or all of those steps in any combination. Selection may be positive (e.g., attaching magnetic beads to T cell markers and washing away other material) or negative (e.g., biochemically, medicinally, or mechanically ablating non-T cells such as tumor cells).

In some embodiments, following an initial expansion, the tumor-specific T cells are selected and further expanded. In a primary, rapid expansion step, high-dose IL-2, anti-CD3, and irradiated allogeneic peripheral blood mononuclear cells (PBMCs) as feeder cells are added to the T cell culture. Various other in vitro expanding and stimulation methods such as cytokines (such as IL-15 and IL-21), costimulatory molecules, immune-checkpoint inhibitors (ICIs), as well as their co-culture with antigen-presenting cells (APCs) or feeder cells may also be used. However, to reduce in vitro culture period and to maintain TILs efficacy, it may be preferable to not perform any TIL selection process and expand the bulk TILs.

The obtained T cells may be expanded, in certain illustrative embodiments, in T-cell medium (Iscove's modified Dulbecco's medium [Life Technologies]) with 7.5% heat-inactivated human serum (Sanquin), 50 U/ml penicillin and streptomycin, and 4 nM glutamin (Lonza) in multi-well plates such as 24-well plates. The T-cell medium may be supplemented with 1.000 IU/ml recombinant human interleukin (IL)-2 (Aldesleukin; Novartis). Wells may be split when the cells at the bottom of a well form a confluent layer or exceeded a concentration of 1.5×10\hat{ }6 TIL/ml.

Expansion methods and protocols may optionally include addition of an IL-2/IL-15/IL-21 cytokine cocktail, which may enhance T cell numbers significantly more than the use of IL-2 alone. T cell expansion during manufacturing may be promoted with PD-1 blockade, 4-1BB stimulation, and CD8+ T-cell enrichment via anti-OX40 antibodies, which are effective strategies not only to improve T cell yield but also to enhance effector cell function of T cells. Addition of an anti-PD-1 antibody to T cell cultures may promote significant increase in the absolute number of T cells and also produce significantly more interferon-γ (IFN-γ) in the presence of an HLA-matched tumor line. Similarly, co-stimulation with agonistic anti-4-1BB antibody may selectively enrich the population of CD8+ cells in the T cell cultures and markedly increase IFN-γ production when compared with an HLA-mismatched tumor line. An agonistic anti-OX40 antibody added to TIL cultures may promote CD8+ TIL expansion at the expense of CD4+ T cells and significantly enhanced IFN-γ secretion compared with untreated T cell cultures, while maintaining the diverse TCR-V(β) repertoire in both CD8+ and CD4+ cell subsets.

Methods may be used to detect specific cell types including, optionally, to detect unwanted cell types (e.g., for removal) such as residual tumor cells. Generally, several techniques, such as allele-specific oligonucleotide real-time quantitative polymerase chain reaction, immunohistochemistry, flow cytometry, and fluorescence in situ hybridization, can be employed to determine residual tumor cells. Using immunohistochemical staining for S100, gp-100, and/or tumor markers such as one or any combination of EpCAM, MART-1, or other know markers, it is possible to detect the presence or absence of residual tumor cells. Exemplary markers that may be used in method of the disclosure are given in Table 1 in Lin, 2021, Circulating tumor cells: biology and significance, Signal Transduction and Targeted Therapy 6:a404, incorporated by reference.

Some embodiments use tumor markers and/or T-cell markers in flow cytometry or FACS to positively select and also remove specific cell types. Methods may be used to remove any residual tumor cells throughout the processes (those removed tumor cells may be kept in a separate culture for a downstream quality check involving introduction to a portion of the final T cell culture to detect anti-tumor activity). Negative selection methods may be employed to remove cells other than desired cells. Negative selection may proceed by biological, mechanical, or other techniques. For example, some embodiments use imaging and physical or laser ablation.

For example, some embodiments use a machine learning/AI algorithm that identifies undesirable cell traits from imaging as cells are cultured/expanded, then apply a laser to ablate the undesirable cells. Cells may be removed by laser ablation, or any related method as discussed in WO 2020/097083 or US 2021/0403942, both incorporated by reference.

Some embodiments utilize a machine learning/AI algorithm that identifies undesirable cell traits from imaging as cells are cultured/expanded and uses the machine learning output to control a laser ablation system to remove unwanted cells. A laser may be used to ablate the undesirable cells. Characteristics of desirability (or lack thereof) might include degree of differentiation or differentiation potential as defined by an ability to differentiate into other cell types, in contrast to T cells that have undergone T cell exhaustion. Some embodiments include a treatment to re-invigorate exhausted T cells. Exhausted CD8+ T cells are characterized by progressive loss of effector functions, high and sustained inhibitory receptor expression, metabolic dysregulation, poor memory recall and homeostatic self-renewal, and distinct transcriptional and epigenetic programs. Exhausted T cells may be reinvigorated through the use of inhibitory receptor blockade, such as αPD-1. See McLane, 2019, CD8 T cell exhaustion during chronic viral infection and cancer, Ann Rev Immunol 47:457-95, incorporated by reference.

In some embodiments, residual tumor cells die out after the culture of cells since the culture conditions only support lymphocytes. In certain embodiments, T cells are filtrated through nylon monofilament mesh to eliminate residual tumor cells. Residual tumor cells may be removed using mononuclear cells stimulated with IL-2 or cultivated in a serum-free environment. Additionally, or alternatively, residual tumor cells can be removed from TIL products using FACS/MACS.

During or after expansion and culturing, e.g., optionally prior to any quality control, packaging, or delivery/administration steps, cells in the culture may be modified. T cells in the culture may be modified to be more active and/or effective. T cells may be edited to have different T cell receptors (TCR) that recognize the tumor. For example, tumor cells or T cells may be subject to nucleic acid sequencing. Sequence may be analyzed to identify tumor targets to TCRs. TCR can be cloned into T cells. Some embodiments include providing T cells with chimeric antigen receptors (i.e., CAR-T cells). In some embodiments, fluid effluent from a situs proximal to a tumor is subject to TCR sequencing to determine sequences of T cell receptors. Those sequences may be analyzed by known methods and antigens may be predicted by that analysis.

The expanded cell products may be subject to quality controls (sterility, negativity for blood-borne diseases, and phenotype checking). Some embodiments include personalized tumor-reactivity assessment phase, which requires the co-culture of TILs with their autologous tumor cells.

The disclosed methods produce products and compositions that are ready to administer to the patient. Preferably before administration, patients undergo lymphodepletion. Using compositions of the disclosure, preferably 10 to 200 billion T cells are administered to the patient optionally along with high dose IL-2. The population of cells is therapeutically useful for treating the tumor. The cells may be administered via the intravenous route, or other routes, such as intrapleural, intraperitoneal, intrathoracic, drain port, or intratumoral, based on the tumor location. Head and neck squamous cell carcinoma (HNSCC) is a heterogenic group of cancers developing from the mucosal epithelium in the oral cavity, pharynx, and larynx. According to causative factors, HNSCC is classified into two categories: human papillomavirus (HPV)-positive and HPV negative cancers. Also, the Epstein-Barr virus (EBV) has been associated with a subtype of HNSCC, so-called nasopharyngeal cancer (NPC). Because viral oncoproteins are expressed in HPV and EBV-associated cancers, HPV and EBV-associated cancers are ideal targets for treatment with compositions of the disclosure. Embodiments of the disclosure may be used to treat HNSCC with a T cell infusion, preceded by standard lymphodepleting chemotherapy, followed by high dose IL-2. Optionally, patients may be given a T cell infusion preceded by ipilimumab and nivolumab, as well as chemotherapy or radiation.

Administration of T cells made by methods of the invention may include co-administration with another therapy. In preferred embodiments, co-administration includes bispecifics.

Melanoma, the fifth leading cancer in the USA (51), develops from the malignant transformation of melanocytes, the pigment-producing cells found in the basal epidermis of the skin, the choroidal layer of the eye, inner ear, and leptomeninges. Methods may include lymphodepletion with cyclophosphamide and fludarabine before T cell re-infusion to promote in vivo clonal expansion of the T cells.

Lung cancer, a leading cause of cancer death globally, includes small cell lung cancer (SCLC) and non-small cell lung cancer (NSCLC) (about ˜90% of all cases). While immune checkpoint inhibitors are used for the treatment of NSCLC, evidence indicates that only a small proportion of patients respond to ICIs, and many of them exhibit immune-related adverse events on immunotherapy. Embodiments may include treating lung cancer (e.g., NSCLC) autologous T cells of the disclosure optional in combination with another treatment such as nivolumab. E.g., a single T cell infusion preceded by standard lymphodepleting chemotherapy, followed by IL-2, and then nivolumab maintenance may be performed.

Ovarian cancer is the eighth major cause of cancer-related mortality in women globally with epithelial ovarian cancer (EOC) being a predominant type. In some embodiments, ovarian cancer is treated with T cell infusion after standard lymphodepleting chemotherapy followed by high doses of IL-2. It is understood that lymphocyte infiltration is associated with a better prognosis in all breast cancer types, especially in triple-negative breast cancer (TNBC) and HER2+ breast cancer. T cell compositions of the invention may be used to treat breast cancer. T cell compositions of the invention may also be used for the treatment of any other suitable cancer type including, for example, colorectal cancer, liver cancer, renal cell carcinoma (RCC), prostate cancer, urothelial bladder carcinoma, and other types of solid tumors, including gastric carcinomas, gynecologic, and urological cancers.

The invention provides methods of preparing a cell therapy by collecting fluid from a site proximal to a tumor and expanding T cells obtained from the fluid. Methods include collecting fluid from a site proximal to a tumor, obtaining at least one T cell from the fluid, and culturing the T cells in vitro. The fluid may be collected during or after surgical removal of a tumor from the site of the surgical removal of the tumor. The fluid may be drain fluid that drains from a surgical site. The method may include diverting the fluid from a waste disposal protocol or vessel in a surgical theater and into a collection vessel. Preferably the fluid includes lymphatic fluid.

What is claimed is:

1. A method of obtaining therapeutic cells, the method comprising:
collecting fluid from a site proximal to a tumor;
obtaining at least one T cell from the fluid; and
culturing the T cells in vitro.

2. The method of claim 1, wherein the fluid is collected during or after surgical removal of a tumor from the site of the surgical removal of the tumor.

3. The method of claim 1, wherein the fluid is drain fluid that drains from a surgical site.

4. The method of claim 1, further comprising diverting the fluid from a waste disposal protocol or vessel in a surgical theater and into a collection vessel.

5. The method of claim 1, wherein the fluid includes lymphatic fluid.

6. The method of claim 1, wherein the obtaining step includes an isolation step.

7. The method of claim 6, wherein the isolation step includes centrifugation, fluorescence-activated cell sorting, capture to magnetic beads, or filtering.

8. The method of claim 1, wherein the culturing step includes an initial expansion with interleukin-2 (IL-2).

9. The method of claim 1, wherein the culturing step includes a primary expansion of T cells in the presence of IL-2 and irradiated allogeneic peripheral blood mononuclear cells (PBMCs) as feeder cells.

10. The method of claim 1, wherein the culturing step includes a primary expansion or an auxiliary expansion that exposes T cells to one or a combination of a cytokine, costimulatory molecule, immune-checkpoint inhibitor, antigen-presenting cells (APCs), PD-1 blockade, 4-1BB stimulation, and agonistic anti-4-1BB antibody.

11. The method of claim 1, wherein the culturing step includes a CD8+ T-cell enrichment using anti-OX40 antibodies.

12. The method of claim 1, wherein the culturing step includes an expansion phase in the presence of an HLA-matched tumor line.

13. The method of claim 1, further comprising a selection step to select T cells.

14. The method of claim 13, wherein the selection step removes tumor cells from culture.

15. The method of claim 13, wherein the selection step uses one or more of laser ablation, flow cytometry, or filtering cells through a membrane.

16. The method of claim 1, further comprising performing a quality control that includes a personalized tumor-reactivity assessment that includes the co-culture of T cells with autologous tumor cells.

17. The method of claim 1, further comprising packaging a product of the culturing step for therapeutic delivery, wherein the product includes millions to billions of T cells.

18. The method of claim 1, further comprising packaging billions of T cells produced by the culturing step in an IV bag.

19. The method of claim 1, further comprising administering T cells produced by the culturing step to a patient from whom the fluid was obtained to treat cancer.

20. The method of claim 19, wherein the administration is intravenous, intrapleural, intraperitoneal, intrathoracic, intrathecal, intratumoral, or via drain port.

21. The method of claim 19, wherein the cancer is head and neck cancer, melanoma, ovarian cancer, breast cancer, colorectal cancer, liver cancer, renal cell carcinoma (RCC), prostate cancer, urothelial bladder carcinoma, gastric carcinoma, gynecologic cancer, or urological cancer.

22. The method of claim 1, wherein the fluid is pleural fluid after lung surgery.
